(19) United States
(12) Patent Application Publication
(10) Pub. No.: US 2026/0076712 A1
(43) Pub. Date: Mar. 19, 2026

(54) Medical Procedure Kit and Methods for Draining Fluid from an Organ

(71) Applicant: CATHETER PRECISION, INC., Fort Mill, SC (US)

(72) Inventors: Dhanunjay LAKKIREDDY, Overland Park, KS (US); David JENKINS, Flanders, NJ (US)

(21) Appl. No.: 19/321,195

(22) Filed: Sep. 6, 2025

Related U.S. Application Data
(60) Provisional application No. 63/695,676, filed on Sep. 17, 2024.

(51) Int. Cl. A61B 17/34; A61M 27/00

(52) U.S. Cl. CPC A61B 17/3417; A61M 27/00; A61M 2205/0238; A61M 2205/32; A61M 2210/122

(57) ABSTRACT

Embodiments include methods for draining fluids from a body organ, such as pericardiocentesis. Embodiments include inserting a large diameter outer needle through the chest wall towards an organ, inserting a smaller diameter microneedle through the outer needle and into the organ, threading a microwire through the lumen of the inner microneedle into the organ, removing the outer needle and inner microneedle, threading a guide sheath over the microwire into the organ, removing the inner dilator and microwire from the guide sheath, inserting a stiffer guide wire through the guide sheath into the organ, removing the guide sheath, threading a drain sheath over the stiffer guide wire into the organ, removing the stiffer guide wire, securing the drain sheath to the patient using a stay fix device, and connecting a suction device to the proximal end of the drain sheath and creating negative pressure to facilitate fluid drainage.

### RELATED APPLICATIONS

This application claims the benefit of priority to U.S. Provisional Patent Application No. 63/695,676 entitled “Medical Procedure Kit and Methods for Draining Fluid from an Organ,” filed Sep. 17, 2024, the entire contents of which are hereby incorporated by reference for all purposes.

### BACKGROUND

Pericardial access and drainage, known as pericardiocentesis, is a medical procedure for managing conditions such as pericardial effusions and cardiac tamponade. Traditional methods involve the use of large-bore needles, which pose significant risks, including trauma to the myocardium, difficulty in visualizing the needle tip, and poor drainage efficiency. The large-bore needles can cause substantial damage to the heart muscle, leading to complications such as bleeding, cardiac tamponade, or arrhythmias. Additionally, conventional methods face challenges in patients with dry pericardium, pectus deformities, or crowded ribs, further complicating access and drainage. Therefore, there is a need for improved methods and apparatus for performing pericardiocentesis that minimize these risks and enhance procedural safety and effectiveness.

### SUMMARY

Some aspects include components and a medical procedure kit of components for draining fluids from an organ, such as the heart, in a pericardiocentesis procedure. Such aspects include a dual needle (needle-in-needle) in which the inner microneedle is sized to minimize injury to the organ, such as when penetrating the pericardium wall. Further aspects include methods for draining fluids from an organ, such as performing pericardiocentesis, using the components of the medical procedure kit.

### BRIEF DESCRIPTION OF THE DRAWINGS

The accompanying drawings, which are incorporated herein and constitute part of this specification, illustrate example embodiments of various embodiments, and together with the general description given above and the detailed description given below, serve to explain the features of the claims.

FIGS. 1A and 1B illustrate a needle-in-needle configuration useful for drawing fluids from an organ according to various embodiments.

FIG. 2 shows a guide sheath with an inner dilator sliding over a microwire.

FIG. 3 shows a stiffer guide wire being pushed through the guide sheath.

FIG. 4 shows the drain sheath with multiple larger holes and a radiopaque tip after being slipped over the stiffer guide wire.

FIGS. 5A and 5B show an example of a stay fix device according to an embodiment.

FIG. 6 illustrates an example of a suction bulb that may be included in the medical procedure kit in some embodiments.

FIG. 7 is a process flow diagram of a method of draining fluids from an organ using the parts within the medical procedure kit.

### DETAILED DESCRIPTION

Various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the claims.

Various embodiments include components, a medical procedure kit, and methods for draining fluids from a patient's organ. In particular, various embodiments support improved needle-in-needle techniques for draining fluids from an organ, such as conducting a pericardium drainage procedure, and a medical kit of the parts for performing organ access and drainage procedures.

Various embodiments are particularly useful for performing pericardiocentesis. The position of the heart close to the chest wall and the toughness of the pericardium wall make reaching and penetrating the pericardium difficult with a single small needle. The needle-in-needle components and technique of various embodiments enable reaching and then penetrating the tough pericardium wall with a needle that is thin enough to minimize trauma to heart tissues. For this reason, various embodiments are described with reference to their use in pericardiocentesis. However, the various embodiments may also be used to drain fluids from other organs, particularly in procedures in which patient outcomes would be improved by the use of a microneedle for penetrating tissues to permit fluid drainage.

A first embodiment includes a medical procedure kit of components designed to work together seamlessly to facilitate safe, efficient, and effective procedures for draining fluids from an organ, such as the heart in pericardiocentesis procedures. The primary components of the medical kit include a large diameter outer needle, a smaller diameter inner microneedle with an outer diameter that fits within the outer needle, a microwire with a diameter that will fit within the microneedle, a guide sheath sized to fit over the microwire and includes an inner dilator; a stiffer guide wire with a diameter that will fit within the guide sheath; a drain sheath sized to fit over the stiffer guide wire and including multiple drain holes; a stay fix device configured to attached the skin of the patient and hold the drain sheath in place; and a drainage bulb or suction device with a connection designed to connect to the drain sheath. The tip portions of the outer and inner needles, the microwire, the guide sheath, and the drawing sheath include a radio-opaque material (e.g., tungsten) to facilitate imaging during the procedure. Each component is designed with specific features to address the limitations of conventional methods, ensuring precise placement, minimal trauma, and efficient drainage.

The large diameter outer needle, typically ranging from 14 to 16 gauge and about 10 to 15 centimeters in length, provides initial stability and directionality. Constructed from high-grade stainless steel, this needle is designed to be used to penetrate the chest wall efficiently, creating a stable path for the inner microneedle. It features a radiopaque marker at the distal end, extending 3-5 millimeters, enhancing visualization under fluoroscopy.

The inner microneedle may be approximately 8 French in size and 15 to 20 centimeters in length and is designed to be introduced through the lumen of the outer needle with a tip and diameter designed for precise penetration of the pericardium with minimal trauma. The microneedle is made of a biocompatible metal and includes a radiopaque marker at its distal tip, extending 3-5 millimeters, allowing for accurate visualization and positioning under fluoroscopy.

The microwire is sized to be threaded through the lumen of the microneedle after the microneedle has penetrated the pericardium. The microwire may be 0.018 to 0.025 inches in diameter and about 150 centimeters in length. Constructed from a combination of stainless steel and a nitinol core, the microwire provides flexibility and strength. The microwire may have a hydrophilic coating at the distal end that can retain a layer of medication to be released in the pericardium and also serve to reduce friction during insertion through the microneedle.

The guide sheath is designed to be threaded over the microwire, may be 4 to 5 French in diameter, and 15 to 20 centimeters in length. Made from flexible yet durable polymer, the guide sheath includes an inner dilator designed to snugly fit the microwire. The distal end of the guide sheath features a radiopaque marker for enhanced visualization under fluoroscopy. In some embodiments, the tip portion of the guide sheath may be coated with or made from a hydrophilic material that can retain a layer of medication to be released in the pericardium.

The stiffer guide wire is sized to be threaded through the guide sheath to provide additional support and stability for the introduction of the drain sheath. This wire may be made from stainless steel with a nitinol core that is approximately 0.035 inches in diameter and 150 to 180 centimeters in length. In some embodiments, the tip portion of the stiffer guide wire may be coated with a hydrophilic material that can retain a layer of medication to be released in the pericardium.

The drain sheath is sized to be threaded over the guide wire into the pericardial space and then draw fluids from within the pericardium. The drain sheath may be about 8 French in diameter and 20 to 30 centimeters in length. Constructed from a flexible polymer, such as polyurethane or silicone, the drain sheath features multiple larger holes along the first 5 to 10 centimeters for efficient drainage and a radiopaque marker at or near the distal end to facilitate imaging during the procedure. The main body of the drawing sheath is stiff to prevent kinking, while the distal tip is designed to be softer than the rest of the sheath to minimize trauma during insertion. The sheath may be coated with a hydrophilic material that can absorb and release medications such as lidocaine, steroids, or anticoagulants.

The stay fix device is designed to secure the drain sheath in place on the patient after insertion and during the drainage procedure. The stay fix device may be about 5-7 centimeters in diameter and constructed from medical-grade adhesive materials and polymers. The stay fix device features a strong adhesive base and options for a Velcro flap or plastic locking mechanism to secure the drain sheath. A circular design and hypoallergenic adhesive may be used to minimize the risk of skin irritation and ensure a secure fit.

The drainage bulb or suction device is made from flexible, medical-grade silicone or latex-free rubber. The drainage bulb or suction device may have a volume capacity of about 100 to 200 milliliters. The bulb design allows for consistent negative pressure when squeezed and released, facilitated by a one-way valve to prevent backflow. The drainage bulb or suction device includes a secure connection port for connecting to the drain sheath and graduated markings for monitoring fluid collection. Alternative designs include an accordion-style drainage device, a vacuum-assisted drainage system, and a syringe-based suction device, each offering specific benefits for different clinical needs.

The invention also includes a medical procedure method for using the kit parts to drain fluids from the pericardium or similar organ structures. The method begins with the introduction of the large diameter outer needle from the xiphisternum towards the heart, parallel to the sternal plate. This needle provides initial stability and directionality. The inner microneedle is then inserted through the lumen of the outer needle and advanced to the pericardium. The microneedle's radiopaque tip allows for precise visualization and positioning under fluoroscopy.

Once the microneedle penetrates the pericardium, a microwire is threaded through its lumen into the pericardial space. The outer needle and microneedle are then withdrawn, leaving the microwire in place. A guide sheath, with an inner dilator, is threaded over the microwire and advanced into the pericardial space. The inner dilator and microwire are removed, and a stiffer guide wire is introduced through the guide sheath into the pericardial space.

The guide sheath is then removed, and the drain sheath is threaded over the stiffer guide wire into the pericardial space. The radiopaque tip and larger holes of the drain sheath ensure efficient drainage and precise placement. Once the drain sheath is positioned, the stiffer guide wire is removed, and the stay fix device is applied to secure the drain sheath to the patient's abdomen. Finally, the drainage bulb or suction device is connected to the proximal end of the drain sheath, and negative pressure is created to facilitate fluid drainage. Various embodiments overcome the foregoing problems by pericardiocentesis.

Various embodiments offer several significant benefits over conventional pericardiocentesis methods. The needle-in-needle technique reduces the risk of trauma to the myocardium and provides enhanced control of the pericardium-penetrating needle during the procedure. The radiopaque markers on the needles, guide wire, and drain sheath improve visualization under fluoroscopy, ensuring accurate placement and reducing the need for repeated adjustments. The multiple larger holes on the drain sheath enhance drainage efficiency, while the hydrophilic coating allows for localized drug delivery, managing pain, reducing inflammation, and preventing infections. The stay fix device ensures stable and secure attachment of the drain sheath, and the drainage bulb or suction device provides consistent and effective negative pressure for fluid removal. These features collectively contribute to safer, more efficient, and more effective pericardial access and drainage procedures. Including all of these parts and supplies in an integrated kit facilitates pericardiocentesis procedures and supports emergency procedures by providing clinicians with all of the tools and supplies needed for the improved procedure.

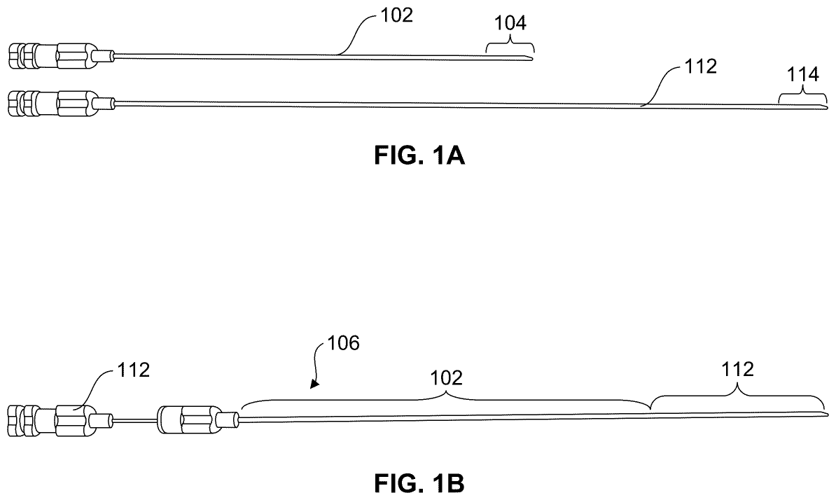

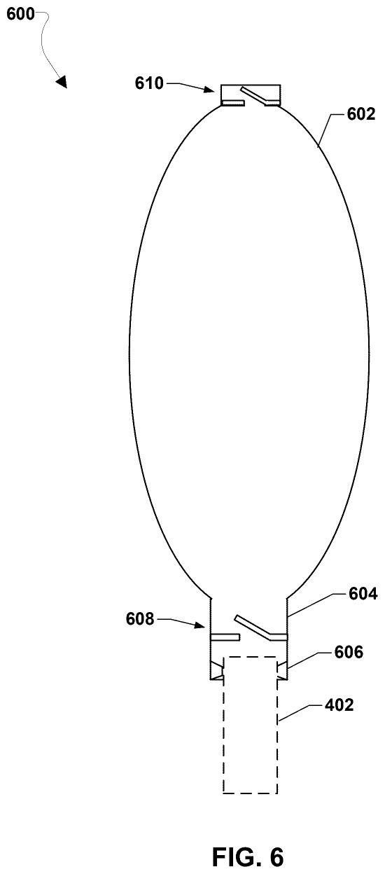

As described, an embodiment includes a medical procedure kit of components for performing a pericardiocentesis procedure. The medical procedure kit includes at least a large diameter outer needle, an inner microneedle, a microwire that will fit within the microneedle, a guide sheath with an inner dilator that will fit over the microwire, a stiffer guide wire that will fit within the guide sheath, a drain sheath that includes multiple drain holes, a stay fix device, and a drainage bulb or suction device with a connection piece for connecting to the drain sheath. Examples of components included in the medical procedure kit are illustrated in FIGS. 1A-6 and described in more detail below with reference to the figures.

FIG. 1A the dual needle components of a larger diameter outer needle 102 and an inner microneedle 112 that is sized to fit within the outer needle. Both needs are components of the pericardial drainage kit.

As described, the larger diameter outer needle 102 provides initial stability and directionality during insertion through the patient's skin and under the chest wall 230 to a position just short of the pericardium wall 232 of the heart. The large-diameter outer needle 102 is designed with patient safety in mind by minimizing trauma during insertion while providing precise control over its placement just outside the pericardial. In some embodiments, this needle has an inner diameter ranging from 12-14 gauge (1) with lengths between 10-15 centimeters (4 inches), allowing for precise control over its placement within various patients of different sizes.

In some embodiments, the large diameter needle 102 may be produced in multiple sizes to accommodate varying patient needs. Shorter needles may be used in pediatric or smaller adult patients, while longer needles may be used in larger adults. The needle's material composition is also adaptable, with stainless steel being a preferred choice due to its strength and durability (2). In some cases, the outer surface of this component may feature hydrophilic coatings for enhanced lubricity during insertion.

In some embodiments, a distal portion 104 of the larger diameter needle 102 may include a radiopaque marker, such as a ring or coating of tungsten or barium sulfate, to provide enhanced visualization under fluoroscopy during the initial insertion procedure. Also, in some embodiments, a portion of the large diameter needle 102 (e.g., on or near the distal end) may include a hydrophilic coating on the surface that can absorb and release medications during insertion, such as a local anesthetic agent (e.g., lidocaine) for pain management during the insertion process.

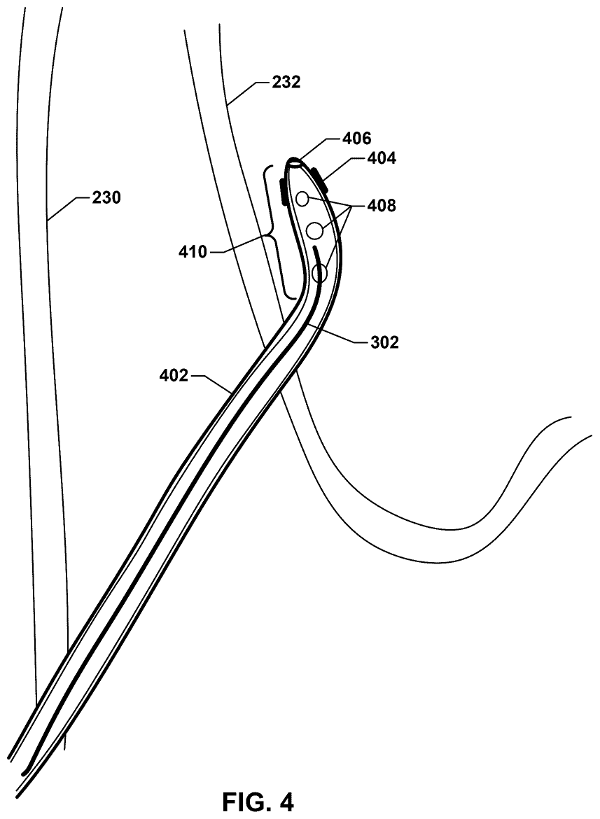

The smaller diameter inner microneedle 112 is sized to pass through the outer needle 102 and then penetrate the pericardial wall 232 to provide an opening through which the drain sheath 402 (FIG. 4) will eventually pass. In some embodiments, the inner microneedle 112 is constructed from a biocompatible metal alloy with a diameter ranging from 0.5 to 1 millimeter (mm) in size. This smaller caliber enables smooth penetration of the pericardium 232 without causing excessive trauma or damage to adjacent tissues.

In some embodiments, a distal portion 114 of the microneedle 112 may include a radiopaque marker, such as a ring or a coating of tungsten or barium sulfate, to provide enhanced visualization under fluoroscopy during insertion through the pericardium wall and into the pericardium volume.

The length of the inner microneedle 112 may vary between 10-20 centimeters to accommodate patients of varying sizes and anatomical configurations, such as shorter needles for pediatric patients and longer needles for large adult patients. In some embodiments, the inner microneedle 112 may be constructed with a flexible tip for improved navigation around curved surfaces within the patient's chest cavity.

In some embodiments, a hydrophilic coating is applied to the surface of the inner microneedle 102 near the distal tip. The hydrophilic coating can absorb and release medications during insertion. Non-limiting examples of the types of medications that may be introduced and introduced to the patient from this coating include local anesthetics (e.g., lidocaine), anti-inflammatory agents (e.g., dexamethasone, triamcinolone acetonide), and anticoagulants (e.g., heparin). This localized medicine delivery mechanism may be beneficial in managing pain, reducing inflammation, or preventing blood clotting within the pericardial space during pericardiocentesis.

FIG. 1B shows the needle-in-needle assembly 106 of the inner microneedle 112 threaded through the outer needle 102 as would occur during a pericardiocentesis procedure. As described, this assembly of two needles allows the clinician to use the stronger outer needle 102 to penetrate the patient's skin, pass under the patient's chest wall 230 and support and position the microneedle 112 near the pericardium wall 232, thereby enabling penetration of this sensitive tissue with a microneedle 112 that would not have sufficient stability to be reliably directed through the patient's skin and tissues all the way to and through the pericardium alone.

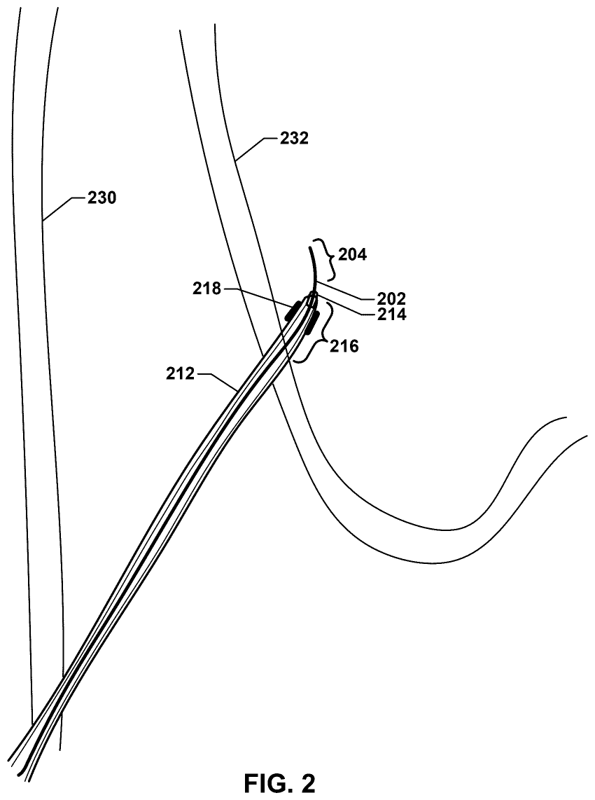

FIG. 2 shows an example of a guide sheath 212 with an inner dilator 214 threaded over the microwire 202 according to some embodiments. As described, the illustrated configuration represents the step of inserting the guide sheath 212 into the pericardium that occurs after the microwire 204 has been threaded through the microneedle 112 and positioned with the distal end 204 within the pericardium volume and the microneedle 112 withdrawn from the patient.

The microwire 202 is a slender wire fabricated of materials that provide flexibility while maintaining stability during navigation through narrow spaces within the patient's tissues. In some embodiments, the microwire 202 may be made of stainless steel with a nitinol core material at its distal end. The nitinol portion may be formed to provide enhanced flexibility and resistance against kinking or buckling when inserted into tight spaces around heart tissues. The microwire 202 may include a radiopaque material, such as tungsten or bismuth sulfate, at or near the distal tip to enhance visibility under fluoroscopy to enable precise control during insertion into the pericardium.

In some embodiments, the microwire 202 may include a hydrophilic coating on or near the distal end 204 made of a material that retains a small amount of medication (e.g., lidocaine or steroids) when dipped into a vial of the medication before insertion into the patient through the inner needle. As this coated surface comes into contact with physiological fluids within the pericardium during insertion, these medications will be released locally to provide targeted therapy for pain management and inflammation reduction.

In some embodiments, the microwire's distal end 204 may be designed with an angled tip of varying degrees (e.g., 10-30°) to facilitate smooth navigation through curved surfaces within the pericardial space. This angled tip, in combination with enhanced visibility during fluoroscopy due to the radiopaque marker, may enable a clinician to precisely guide the microwire 202 within heart tissues and the pericardium volume.

In some embodiments, the microwire 202 may be provided in different lengths to accommodate patients of different sizes and procedure requirements. For instance, shorter wires (e.g., 10-15 cm) may be used in pediatric or small adult patients, while longer wires (20-30 cm) may be used for large adults.

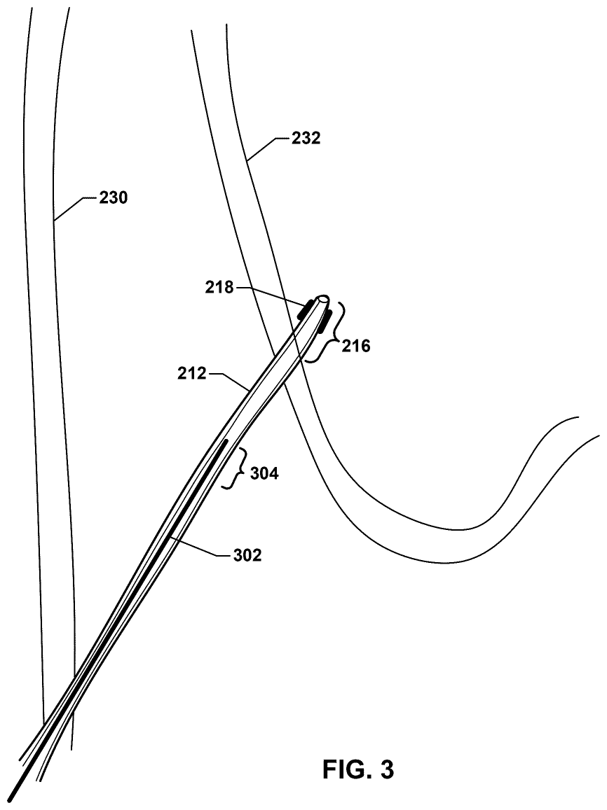

The guide sheath 212 with the inner dilator 214 enables replacing the microwire 202, which is sized to pass through the inner needle 212, with the stiffer guide wire 302 (FIG. 3) that is strong enough to guide the drain sheath 402 (FIG. 4) into the proper location in the pericardium. The combination of the microwire 202 followed by the guide sheath 402 enables precise navigation through narrow spaces within the pericardium while enabling guided insertion of the stiffer guide wire 302 that is used to guide the insertion of the drain sheath 402.

The guide sheath 212 features an outer layer constructed from flexible yet durable polymer materials such as polyurethane or silicone, which provides flexibility for smooth passage over the microwire 202 without compromising structural integrity. The distal end 216 of this component features a radiopaque marker 218 made from materials such as tungsten or barium sulfate for enhanced visualization under fluoroscopy during the insertion procedure. The guide sheath 212 with the inner dilator 214 may have a diameter between 4 and 6 French and have an inner diameter to accommodate guidewires of different diameters (e.g., 0.018-0.025 inches). In medical procedure kits for performing pericardiocentesis on pediatric patients and smaller adults, the guide sheath 212 with inner dilator 214 features shorter length options ranging from 10-15 centimeters, while the guide sheath 212 may be up to 20 cm in length in kits for pericardiocentesis procedures on large adult patients.

In some embodiments, the guide sheath 212 may include a hydrophilic or other coating on the distal portion 216 and/or other segments to facilitate medication release within the pericardial space. Like the other components in the medical procedure kit, this coating enables localized delivery of therapeutic agents directly into tissues surrounding the drainage site, thereby enhancing treatment outcomes for patients undergoing cardiac tamponade or other conditions.

In some embodiments, the guide sheath 212 may include a soft or collapsible material near the distal end 216 and/or inner dilator 214 to reduce trauma to tissues when penetrating the pericardium wall 232. In some embodiments, portions of the guide sheath 212 may feature an accordion-style design (not shown) in which multiple segments of varying stiffness levels are integrated along a portion of the guide sheath. This configuration may facilitate navigating the sheath around curved surfaces within the patient, such as under the chest wall 230.

FIG. 3 shows the stiffer guide wire 302 being threaded through the guide sheath 212 according to some embodiments. As described, the stiffer guide wire 302 has sufficient rigidity to maintain its shape as the guide sheath 212 is withdrawn and the drain sheath 402 is passed over it, ensuring precise placement of the drain sheath 402 within the pericardium volume. Radiopaque materials may be included near the distal portion 304 of the wire 302 to facilitate imaging via fluoroscopy during the insertion procedure.

In some embodiments, the stiffer guide wire 302 may be made from stainless steel with a nitinol core material to provide flexibility and strength. The outer diameter of the wire ranges between 0.035 inches (1 mm) and 0.045 inches (2 mm), allowing for precise control during insertion while minimizing tissue trauma. In some embodiments, multiple segments with different stiffness properties may be integrated into the stiffer guide wire 302 to facilitate navigation around curved surfaces within the patient.

Like other components in the medical procedure kit of various embodiments, the stiffer guide wire 302 may include a hydrophilic coating applied to the distal end 304 to enable the release of medication when the surface area contacts physiological fluids during insertion and positioning.

In some embodiments, an additional layer of material having varying levels of stiffness properties may be integrated into the stiffer guide wire 302 for specific clinical applications where enhanced support is required. This may include a more rigid segment near its distal end 304 to provide added stability while navigating complex anatomical structures or maintaining shape during insertion and positioning of the drain sheath 402.

Like other components in the medical procedure kit, the stiffer guide wire may be provided in different lengths to support procedures on different sized patients.

FIG. 4 shows the drain sheath 402 being threaded over the stiffer guide wire 302. As described, the drain sheath 402 includes a radiopaque tip 404 (e.g., a ring or layer of tungsten or barium sulfate) to enhance visualization under fluoroscopy, facilitating accurate placement and monitoring within the pericardial space during insertion and pericardiocentesis. The drain sheath 402 includes multiple holes 406, 408 along a distal portion 410 (e.g., a 5-10 centimeter section) of its length, with the holes sized and positioned to facilitate fluid collection during pericardiocentesis. In some embodiments, these holes are evenly spaced at approximately 1 millimeter apart to ensure consistent drainage patterns across the entire surface area in the distal portion 410.

In some embodiments, the drain sheath 402 is constructed from flexible yet durable polymers, such as polyurethane or silicone, providing a soft and pliable material that minimizes tissue trauma during insertion.

In some embodiments, a hydrophilic coating is applied to specific sections of the drain sheath surface, such as within the distal portion 410, that will be positioned within the pericardium or along its entire length. This coating is provided to allow localized delivery of medications such as lidocaine, steroids, or anticoagulants. This mechanism enables targeted therapy with reduced systemic side effects during pericardiocentesis procedures.

In some embodiments, the drain sheath 402 may have an accordion-style design (not shown) over portions of the length with multiple segments of varying stiffness levels to facilitate navigation around curved surfaces during insertion and to reduce pressures applied to tissues during fluid collection.

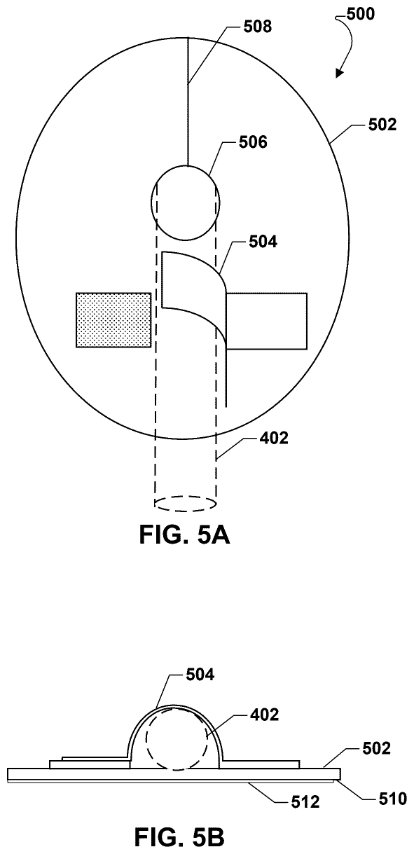

FIGS. 5A and 5B illustrate an example configuration of a stay fixed device 500 that may be included in the medical procedure kit according to some embodiments. The various designs of the stay fix device 500 offer flexibility in terms of material selection, size adjustment, or locking mechanisms. This adaptability enables customization for specific clinical applications while providing a secure attachment mechanism that ensures effective drainage while minimizing patient discomfort.

As explained, the stay fix device 500 is configured to secure the proximal end of the drain sheath 402 in place on the patient's abdomen after insertion. In some embodiments, the stay fix device 500 may have an elliptical or circular base 502 with hypoallergenic adhesive materials on an underside 510 (FIG. 5B) used to ensure stable attachment of the device to the patient while minimizing skin irritation risks. A removable protective plastic sheet or strip 512 may be applied to the adhesive materials to protect the adhesive, with the sheet or strip 512 removed just prior to placement on the patient.

In some embodiments, the stay fix device 500 includes a flexible base 502 made from polyurethane foam and polyester fibers to provide flexibility to comfortably conform to the patient's anatomy. The elliptical or circular design of the base 502 allows it to distribute pressure evenly across a large surface area on the patient's abdomen, reducing discomfort or irritation caused by uneven attachment.

The stay fix device 500 incorporates a locking mechanism 504 that secures the drain sheath 402 in place on the base 502. Such a locking mechanism 504 may include features to provide adjustable tension control to ensure sufficient attachment force on the drain sheath 402 while minimizing patient discomfort. In the embodiment illustrated in FIGS. 5A and 5B, the stay fix device 500 includes a Velcro® flap locking mechanism 504 that the clinician can use to secure the drain sheath 402 in place without placing pressure on the patient's skin. This design allows healthcare professionals to adjust tension control during application, ensuring optimal fixation and minimizing patient discomfort. However, the illustrated example of a Velcro® flap is only one possible implementation of a locking mechanism 504. Other mechanisms may be used without departing from the scope of the claims, such as a plastic locking mechanism that folds over the drawing sheath 402 and clips into a latch (not shown).

In some embodiments, the stay fix device 500 includes multiple locking mechanisms 504 (e.g., Velcro® flaps) arranged in a pattern on the base 502 to provide redundant or greater retention force and/or accommodate different incision locations, different patient body types, and drain sheath profiles. Such embodiments may enable attachment at various points along the drain sheath 402 to provide flexibility in securing the drain sheath to the patient. In such embodiments, each locking mechanism 504 may be adjusted independently of others to optimize pressure distribution and minimize discomfort. In some procedures, multiple stay fix devices 500 may be used to secure the drain sheath 402 over an extended portion of the patient's body. Therefore, the medical procedure kit may include more than one stay fix device 500.

In some embodiments, the base 502 may include an opening 506 sized and shaped to accommodate the drain sheath 402, allowing the sheath to pass through the base 502 when attached to the patient. In some embodiments, the base 502 may also include an opening or slit 506 that can be pulled apart to facilitate placement of the drain sheath 402 in the opening 504 without having to thread the stay fix device 500 over the sheath. This is an optional configuration, and in some embodiments the base 502 may not include an opening 504, and instead, the drain sheath 402 may be positioned to lie on top of the base 502 and be secured solely by the locking mechanism 504.

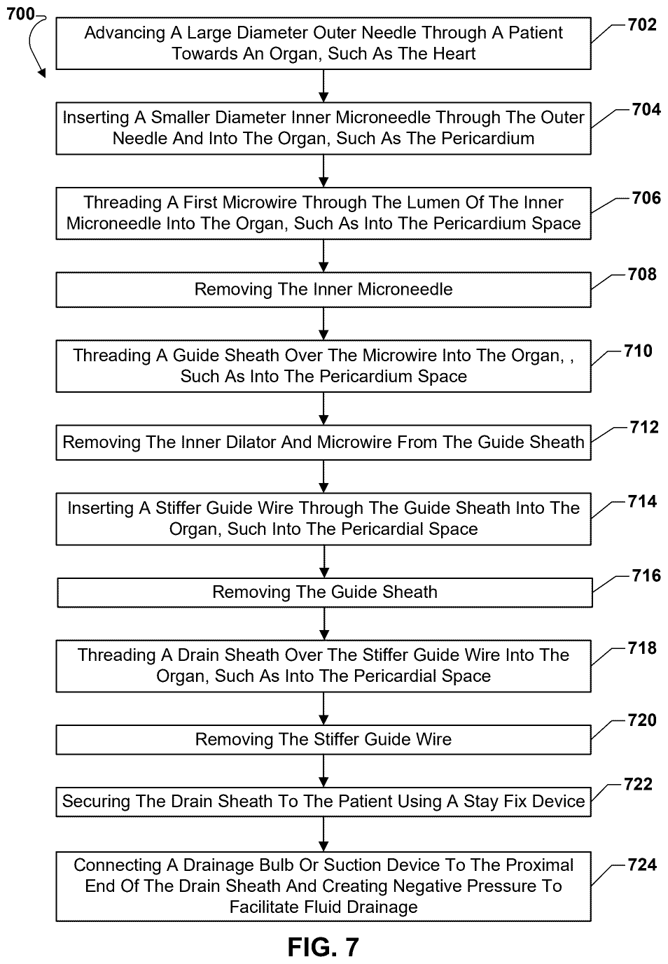

FIG. 7 illustrates an example of a drainage bulb 600 that may be included in the medical procedure kit to serve as a suction device to facilitate fluid removal from around the heart during pericardiocentesis procedures. The drainage bulb 600 or suction device enables applying negative pressure to the drain sheath to facilitate fluid collection while minimizing backflow risks. The drainage bulb 600 may include a flexible bulb 602 made of a variety of materials, including flexible silicone or latex-free rubber. In some embodiments, the bulb 602 may be made from transparent or translucent plastic and include graduated markings (not shown) on the exterior surface to facilitate monitoring and tracking of fluid collection volumes during pericardiocentesis procedures.

The bulb 602 may be coupled to a connector portion 604 that includes a connection mechanism 606 or a snap-fit design for coupling the drainable bulb 600 to a drain sheath 402. The bulb 602 may incorporate (e.g., within the connector portion 604) a one-way valve 608 configured to prevent the backflow of fluids into the drainage bulb 602 when the bulb is squeezed by the clinician. The bulb 602 may also include a one-way valve 610 configured to permit air to be expelled when the bulb is squeezed and prevent air from reentering the bulb when it is released. The two one-way valves 608, 610 permit the creation of a suction force applied to the drain sheath 402 by squeezing and then releasing the bulb.

In some embodiments, the drainage bulb 600 features an accordion-style design (not shown) with multiple segments that compress and expand in response to changes in negative pressure. This configuration allows for consistent fluid collection while reducing resistance during compression cycles.

In some embodiments, a vacuum-assisted system may be used instead of a drainage bulb to generate suction through a separate vacuum pump (not shown) connected via tubing to the drain sheath 402.

In some embodiments, a syringe-based suction device (not shown) may be used and included in the medical procedure kit instead of a traditional bulb. In such embodiments, the piston in the syringe generates negative pressure during fluid collection. Such embodiments may enable precise control over vacuum levels while minimizing backflow risks through calibration of the syringe's plunger.

In some embodiments, an integrated drainage bulb or suction device may be made with a collapsible design that can be compressed when not in use to reduce bulkiness for easier storage within the medical procedure kit.

FIG. 7 illustrates an example method for draining fluids from an organ, such as performing pericardiocentesis, using the components of the medical procedure kit according to some embodiments. With reference to FIGS. 1A-7, the method 700 may be performed by a clinician using the components of the medical procedure kit as described and claimed herein. Means for performing functions of the method 700 may include components of the medical procedure kit as described and illustrated.

In block 702, the clinician may perform operations including advancing a large diameter outer needle 202 into the patient and towards the organ to be drained. In a pericardiocentesis procedure, this may include advancing the outer needles beneath the chest wall towards the pericardium of the patient's heart. During insertion, the clinician may track the location and movement of the needle via fluoroscopy with images enhanced by the radiopaque marker at its distal end. In some embodiments, a medication may be applied to the distal portion of the outer needle, such as by dipping a hydrophilic portion near the distal end of the needle into a vial of the medication.

As this needle does not penetrate the organ (e.g., the pericardium), it may include a large outer diameter (typically ranging from 12-14 gauge) with a length of approximately 10 to 15 centimeters. The patient's skin is prepared by cleaning it thoroughly before the procedure and applying local anesthesia as needed for comfort during insertion. In some cases in which access is limited due to scarring or other factors, a curved outer needle (e.g., approximately 15 degrees angle between its longitudinal axis) may be used for improved navigation through narrow spaces in the patient's chest cavity.

In a pericardiocentesis procedure, the insertion process typically begins with the operator positioning themselves in front of the patient and aligning their body parallel to that of the subject. The chest wall is then stabilized by applying gentle pressure or using a stabilizing device, allowing for precise control during needle placement. Once properly positioned, the outer needle is inserted through the skin at an angle of approximately 30-40 degrees relative to its longitudinal axis in order to minimize trauma and discomfort.

In block 704, the clinician may perform operations including inserting a smaller diameter inner microneedle 212 through the outer needle 202 and into the organ (e.g., the pericardium). As described, the inner microneedle may be 8 French in diameter and provided in different lengths for use with different sized patients, such as pediatric or adult patients. In some embodiments of the inner microneedle may incorporate a hydrophilic coating at their distal ends, and as part of the operations in block 704, the clinician may apply a small amount of medication to this coating (e.g., dipping in a vial of the medication), such as lidocaine or steroids before insertion into the outer needle.

In block 706, the clinician may perform operations including threading a first microwire through the lumen of the inner microneedle and guiding it into the organ (e.g., into the pericardial space). Threading the microwire through the inner microneedle begins by introducing the distal end of the micro-wire into the lumen of the inner microneedle. The clinician may monitor the location and direction of advance of the microneedle by viewing the microwire, and particularly the tip portion radiopaque marker, using fluoroscopy during insertion.

In some embodiments, a hydrophilic coating at the distal end of the microwire is configured to retain and release medications that may be beneficial for organ (e.g., pericardial) drainage procedures. As part of the operations in block 706, the clinician may apply a small amount of medication to this coating (e.g., dipping in a vial of the medication), such as lidocaine or steroids, before insertion into the microneedle.

During the insertion process in block 706, the clinician may turn and advance the microwire after the distal end extends beyond the tip of the microneedle so as to position the end of the microneedle in a desired location for the drain sheath in a later step. Again, the clinician may monitor the position, orientation, and direction of advancement of the microneedle using fluoroscopy enhanced by a radiopaque marker at the distal end.

In block 708, the clinician may perform operations including removing the inner microneedle while leaving the microwire in place. In some embodiments, both the outer and inner needles may be withdrawn in a controlled manner to minimize trauma or discomfort for the patient and without withdrawing the microwire. In some embodiments, the clinician may first withdraw the microneedle from the outer needle while maintaining insertion pressure on the microneedle, followed by removal of the outer needle. In some embodiments, the clinician may remove both needles in the same withdrawal motion. In some embodiments, only the inner microneedle may be removed, leaving the larger outer needle in place to facilitate positioning the guide sheath and then the drain sheath in the pericardium. In such embodiments, the outer needle may be removed after the drain sheath is in position.

In block 710, the clinician may perform operations including threading a guide sheath over the microwire into the organ, such as into the pericardial space. The guide sheath is configured to be threaded over the microwire so as to follow the microwire through the pericardial wall and into a location where fluids will be drained. During insertion, the clinician may monitor the location and direction of advance of the guide sheath by viewing the radiopaque marker near the distal end using fluoroscopy.

In some embodiments, the distal end of the guide sheath may include a hydrophilic coating that may absorb medication to be released into the pericardial space. In such embodiments, as part of the operations in block 710, the clinician may apply a small amount of medication to this coating (e.g., dipping in a vial of the medication), such as lidocaine or steroids, before insertion into the patient.

In block 712, the clinician may perform operations including removing the inner dilator and microwire from the guide sheath. Once the guide sheath is in place within the pericardium space, the clinician may withdraw the inner dilator and the microwire from within the lumen of the guide sheath by retracting them in an axial direction while maintaining a secure grasp on the guide sheath.

In block 714, the clinician may perform operations including inserting a stiffer guide wire through the guide sheath into the organ, such as into the pericardial space. This may be accomplished by pushing the guide wire into the guide sheath while maintaining a secure grasp on the guide sheath. During insertion, the clinician may monitor the location and direction of advance of the guide wire by viewing the radiopaque marker near the distal end using fluoroscopy.

In some embodiments, the distal end of the stiffer guide wire may include a hydrophilic coating that may absorb medication to be released into the pericardial space. In such embodiments, as part of the operations in block 714, the clinician may apply a small amount of medication to this coating (e.g., dipping in a vial of the medication), such as lidocaine or steroids, before insertion into the guide sheath.

In block 716, the clinician may perform operations including removing the guide sheath. Once the guide wire is in position within the pericardium, the clinician may withdraw the guide sheath from the patient by pulling on it while maintaining a secure grasp on the guide wire to prevent it from being removed.

In block 718, the clinician may perform operations including threading a drain sheath over the stiffer guide wire into the organ, such as into the pericardial space. As described, the drain sheath includes multiple holes at the distal end through which fluids in the organ may be drained. Insertion of the drain sheath may involve threading the drain sheath over the guide wire and pressing it into the patient while maintaining a secure grasp on the guide wire to prevent movement of the wire. During insertion, the clinician may monitor the location and direction of advance of the drain sheath by viewing the radiopaque marker near the distal end using fluoroscopy.

In some embodiments, the distal end of the drain sheath may include a hydrophilic coating that may absorb medication to be released into the pericardial space. In such embodiments, as part of the operations in block 718, the clinician may apply a small amount of medication to this coating (e.g., dipping in a vial of the medication), such as lidocaine or steroids, before insertion into the patient.

In block 720, the clinician may perform operations including removing the stiffer guide wire. Once the drain sheath is in place within the organ, such as within the pericardium space, the guide wire is removed so that fluid can be drawn through the sheath. The clinician may withdraw the guide wire from the patient by pulling on it while maintaining a secure grasp on the drain sheath to prevent it from being removed.

In block 722, the clinician may perform operations including securing the drain sheath to the patient using a stay-fix device. These operations may include removing a protective sheet or strip from the adhesive layer on the underside of the stay-fix device and then applying it to the patient near the incision where the drain sheath exits the patient's body. Once applied, the drawing sheath may be secured to the stay-fix device using the locking mechanism (e.g., a plastic strap or Velcro® flap) so as to hold the sheath in place with respect to the incision site.

In block 724, the clinician may perform operations including connecting a drainage bulb or suction device to the proximal end of the drain sheath and creating negative pressure to facilitate fluid drainage. As described, the drainage bulb or suction device may include a connection mechanism into which the drain sheath is coupled to form an air and fluid type coupling. Suction operations may then be applied by the clinician by squeezing the drainage bulb to create a suction force in the drain sheath. In embodiments in which other suction is applied to the drain sheath by other means, such as a syringe-based suction device or a separate vacuum pump, the operations in block 724 may include connecting the drain sheath and then activating the device to apply suction force.

The preceding medical procedure method descriptions and the process flow diagrams are provided merely as illustrative examples and are not intended to require or imply that the operations of the various embodiments must be performed in the order presented. For example, the stay fix device may be applied to the patient at any time during the procedure and may be used to secure components other than the drain sheath during different operations, such as stabling the outer needle during insertion of the inner needle, stabilizing the guide sheath during removal of the microwire, and/or stabilizing the drain sheath during removal of the stiffer guide wire.

The preceding medical procedure method descriptions are not intended to encompass all operations that will be performed during a procedure as normal surgical practices and precautions are not described so as to focus on the points of novelty. One of skill in the art would appreciate that more components may be included in the medical procedure kit and used at various stages in the procedures, such as antiseptics, wound closure tools and supplies, and the like.

Again, while the various embodiments are described in terms of performing pericardiocentesis, the components, medical procedure kit, and procedures of various embodiments may be applied in a similar manner to drain fluids from other body parts and organs. Therefore, references to pericardiocentesis procedures are not intended to limit the scope of the claims unless specifically recited in a claim.

As will be appreciated by one of skill in the art, the order of operations in the preceding embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an,”or “the”is not to be construed as limiting the element to the singular.

The preceding description of the disclosed embodiments is provided to enable any person skilled in the art to make or use the claims. Various modifications to these embodiments will be readily apparent to those skilled in the art, and the generic principles defined herein may be applied to other embodiments and implementations without departing from the scope of the claims. Thus, the present disclosure is not intended to be limited to the embodiments and implementations described herein but is to be accorded the widest scope consistent with the following claims and the principles and novel features disclosed herein.

What is claimed is:

1. A medical procedure kit for draining fluids from a body organ, comprising:
a large diameter outer needle;
a smaller diameter inner microneedle with an outer diameter that fits within the outer needle;
a microwire with a diameter that will fit within the microneedle;
a guide sheath sized to fit over the microwire and includes an inner dilator;
a stiffer guide wire with a diameter that will fit within the guide sheath;
a drain sheath sized to fit over the stiffer guide wire and including multiple drain holes;
a stay fix device comprising an adhesive portion for attaching to the skin of the patient and a securing mechanism configured to hold the drain sheath in place; and
a drainage bulb or suction device with a connection piece designed to connect to the drain sheath.

2. The medical procedure kit of claim 1, wherein the outer needle and inner microneedle have radiopaque markers at their distal tips.

3. The medical procedure kit of claim 1, wherein the drain sheath includes multiple larger holes distributed along the first 5 to 10 centimeters from the distal end.

4. The medical procedure kit of claim 1, wherein a distal portion of the drain sheath includes a hydrophilic coating that can absorb and release medications.

5. The medical procedure kit of claim 1, wherein the stay fix device securing mechanism is a Velcro flap, a plastic locking mechanism, or a combination of both.

6. The medical procedure kit of claim 1, wherein the drainage bulb or suction device includes a one-way valve to prevent backflow of fluid.

7. The medical procedure kit of claim 1, wherein the drainage bulb or suction device includes graduated markings for monitoring fluid collection.

8. A method for draining fluids from an organ, comprising:
advancing a large diameter outer needle in a patient toward the organ;
inserting a smaller diameter inner microneedle through the outer needle and into the organ;
threading a first microwire through the lumen of the inner microneedle into the organ;
removing the outer needle and inner microneedle;
threading a guide sheath over the microwire into the organ;
removing the inner dilator and microwire from the guide sheath;
inserting a stiffer guide wire through the guide sheath into the organ;
removing the guide sheath;
threading a drain sheath over the stiffer guide wire into the organ;
removing the stiffer guide wire;
securing the drain sheath to the patient using a stay-fix device; and
connecting a drainage bulb or suction device to the proximal end of the drain sheath and creating negative pressure to facilitate fluid drainage.

9. The method of claim 8, wherein:
the method is a method of pericardiocentesis;
the organ is a heart;
the microneedle penetrates the pericardium;
the microwire, guide sheath, stiffer guide wire, and drain sheath are advanced into the pericardium space; and
fluid is drained from within the pericardium.

10. The method of claim 8, wherein the outer needle and inner microneedle have radiopaque markers at their distal tips.

11. The method of claim 8, wherein the drain sheath has multiple larger holes distributed along the first 5 to 10 centimeters from the distal end.

12. The method of claim 8, wherein the drain sheath includes a hydrophilic coating that can absorb and release medications.

13. The method of claim 8, wherein the stay fix device comprises an adhesive base and a securing mechanism selected from the group consisting of a Velcro flap and a plastic locking mechanism.

14. The method of claim 8, wherein the drainage bulb or suction device includes a one-way valve to prevent backflow of fluid.

15. The method of claim 8, wherein the drainage bulb or suction device includes graduated markings for monitoring fluid collection.
